(19) United States
(12) Patent Application Publication
(10) Pub. No.: US 2021/0240853 A1
(43) Pub. Date: Aug. 5, 2021

(54) DE-IDENTIFICATION OF PROTECTED INFORMATION

(71) Applicant: KONINKLIJKE PHILIPS N.V., EINDHOVEN (NL)

(72) Inventors: Eric Thomas Carlson, New York, NY (US); Mohammad Shahed Sorower, Natick, MA (US); Sreramkumar Sitaraman Viswanathan, Bangalore (IN); Sreekanth Manakkaparambil Sivanandan, Bangalore (IN); Anshul Jain, Bangalore (IN); Sunil Ranjan Khuntia, Bangalore (IN); Ze He, Cambridge, MA (US)

(73) Assignee: KONINKLIJKE PHILIPS N.V., EINDHOVEN (NL)

(21) Appl. No.: 17/267,523

(22) PCT Filed: Aug. 23, 2019
(86) PCT No.: PCT/EP2019/072562

§ 371 Date: Feb. 10, 2021

Related U.S. Application Data
(60) Provisional application No. 62/723,534, filed on Aug. 28, 2018.

(51) Int. Cl. G06F 21/62; G16H 10/60; G16H 10/20; G06N 20/00

(52) U.S. Cl. CPC G06F 21/6254; G06N 20/00; G16H 10/20; G16H 10/60

(57) ABSTRACT

The present disclosure is directed to methods and apparatus for centralized de-identification of protected data associated with subjects. In various embodiments, de-identified data may be received (1102) that includes de-identified data set(s) associated with subject(s) that is generated from raw data set(s) associated with the subjects. Each of the raw data set(s) may include identifying feature(s) that are usable to identify the respective subject. At least some of the identifying feature(s) may be absent from or obfuscated in the de-identified data. Labels associated with each of the de-identified data sets may be determined (1104). At least some of the de-identified data sets may be applied (1108) as input across a trained machine learning model to generate respective outputs, which may be compared (1110) to the labels to determine a measure of vulnerability of the de-identified data to re-identification.

### TECHNICAL FIELD

Various embodiments described herein are directed generally to de-identification of protected data. More particularly, but not exclusively, various methods and apparatus disclosed herein relate to scalable de-identification of protected data in various contexts.

### BACKGROUND

As technology advances, more and more data is being collected, e.g., from the “internet of things,” as well as from more specialized data sources such as health care equipment and personnel. For example, with the advent of the Electronic Health Record (“EHR”) system, there is an exponential growth in the volume of information (e.g., symptoms, diagnoses, procedures, medications etc.) collected from patients during the course of a treatment. A multi-specialty hospital has many departments resulting in the generation of hundreds of gigabytes of data every day. Also, more and more structured data is being made available for research. As data collection and proliferation becomes more and more ubiquitous, it becomes increasingly important to anonymize various types of protected data while also allowing the data to be leveraged to its full potential. For example, various types of data may be subjected to de-identification or anonymization processing in which data that are usable to identify an individual or group may be scrubbed while other data may be maintained in some form so that it can be used for various beneficial purposes.

Patient healthcare data can be extremely useful for a variety of purposes, such as disease research, development of drugs and other treatments, etc. However, this data is typically considered highly sensitive, and therefore may be covered by national, regional, hospital, or business regulations. Examples include the Health Insurance Portability Act (“HIPAA”) requirements for data privacy in the US, Informatics for Integrating Biology and the Bedside (“i2b2”), Medical Information Mart for Intensive Care (“MIMIC”), business-to-business master research agreements, agreements stipulated by institutional review boards, and so forth. Each set of regulations may impose or alter requirements for how patient healthcare data is handled. This particularly applies to de-identification, in which protected health information (“PHI”) is identified and either modified (e.g., obfuscated) or removed in order to limit risk to patients and care providers. HIPAA lists eighteen such PHI elements that specifically must be removed for a dataset to be considered “de-identified”under that standard. Other agreements or regulations may identify more or fewer elements, or may allow for the elements to be transformed rather than removed, balancing research requirements and other privacy safeguards with re-identification risk.

### SUMMARY

Given the many possible requirements for what constitutes PHI in a particular study or how that PHI is required to be handled, efforts to create a software system capable of producing de-identified output acceptable to all standards have failed. Instead, software systems have been created piecemeal that are tailored for each application. The problem is compounded by the requirement to process many different types of data, such as imaging data, electronic medical record (“EMR”) extracts, waveforms, free text notes, etc., in a consistent manner such that the output of all systems may be linked to form a full multi-modal view of the patient. The traditional solution to this problem has been to create individual software systems that process each type of data, as well as each modality of a data type. Each new type of data to be processed requires re-implementation of the de-identification components, consistent configurations to ensure that all components are treating PHI in an identical way, and methods of ensuring that the output of each isolated processing layer is consistent. This is especially difficult if look-up tables are required (as they often are), and lookup tables must be synced between processing components.

Accordingly, the present disclosure is directed to a framework for centralized de-identification of protected data associated with subjects in multiple modalities based on a hierarchal taxonomy of policies and corresponding handlers (e.g., micro-services, functions, etc.), as well as techniques for scaling de-identification processes for large datasets, progressive de-identification, and de-identification verification (i.e. leakage detection).

For example, in the healthcare context, techniques described herein may be implemented to provide a centralized platform that is capable of processing multiple data streams containing multiple data types and/or data modalities. The platform may be easily configurable to perform de-identification in accordance with a variety of different regulations, as well as to facilitate other features such as deduplication, auditing, and/or discoverability. In some embodiments, the platform may make use of a hierarchal taxonomy to classify individual data points, as well as to select handlers to process the data points in accordance with their classifications. Techniques disclosed herein create a single software platform and framework to act as a single point of configuration and to perform centralized PHI de-identification for all processing modalities. A flexible configuration syntax is described that can cover HIPAA and other use cases, and be extended as needed to localized requirements. All modality-specific components make use of this central service, ensuring that the outputs are consistently de-identified to meet regulatory requirements and to facilitate creation of a multi-modal linked dataset. Techniques described herein are also applicable in a de-centralized nature. For example, an individual computing device (or computing devices of a remote site, such as a doctor's office) may be configured to perform selected aspects of the present disclosure. The centralized service also facilitates scaling to large datasets, load balancing, progressive de-identification, and leakage detection.

As used herein, a “data type” refers to a type of data, e.g., a source of data. One example of a data type is a subject identifier. Subject identifiers can include what will be referred to herein as “external,” “internal,” and “system” identifiers. An external identifier is a general-purpose identifier (although it may have been initially created for a specific context) that is used in a variety of circumstances beyond a particular context, such as a social security number, a driver's license number, United States Veterans Affairs account number, and so forth. An internal identifier, by contrast, is limited to a particular context. In the healthcare context, internal identifiers may be used within hospital information systems to identify patients, and may include, for instance, a medical record number or a hospital encounter identifier, and are typically available to healthcare personnel and perhaps even patients. A system identifier (e.g., a database row id) is used exclusively in a software/database system and is typically not made available outside of that system (e.g., it is not “surfaced” to patients or medical personnel). Other data types include, but are not limited to, age, contact (e.g., telephone number, email, IP address), datetime (any date and/or time, such as a subject's birthdate, date of admittance, date of treatment, etc.), location (e.g., zip code, street address, state, city, etc.), name (e.g., given name, family name), “no-PHI” (any value known not to be PHI under any definition, such as heart rate), and organization or “org” (e.g., hospital name, name of study or trial, name of study or trial sponsor, etc.).

As used herein, a “data modality” or “modality” refers to a way of expressing a particular data type, e.g., with a particular level of granularity. For example a datetime can be expressed in a number of ways (i.e. modalities), such as ISO 8601. As another example, a location data type can be expressed in various modalities and/or granularities, such as a ZIP code, a street address, a city/state, etc. As yet another example, phone numbers may be expressed in various ways, such as with or without area codes, with or without interspersed commas, and so forth. In various embodiments, various modalities may be captured by regular expressions or other similar means.

As used herein, “structured data” is a broad category that covers many types of data that may be processed using techniques described herein. Structured data may include, but is not limited to, EMR extracts, medical/insurance claims, Fast Healthcare Interoperability Resources (“FHIR”), and HL7 data. As will be described below, in some embodiments, each of these sub-categories may have its own de-identification processor, although this would result in code duplication, differing features in different processors, and potentially different processing capabilities in different processors. Accordingly, in some embodiments, techniques described herein facilitate a data processing pipeline that leverages a generalized structured data processor. The schema of an input may be provided along with the data itself, with the schema being used to locate and process the protected data (e.g., PHI). In this way, schemas have been created for several FHIR resource classes, Cerner tables, and claims tables, and can easily be created to allow for processing of new data structures as needed.

As used herein, “free text data” can come from a variety of sources, including discharge summaries, radiology reports, nurse progress notes, or family notes. Such notes would generally be contained within structured data, so a data processing module configured to process free text data can be called either independently or by the structured data processor. In some implementations, initial processing may be based on the rule-based MIMIC Freetext De-identification Tool, used by Physionet to create the MIMIC notes repository. Additional processing options may include recurrent neural network (“RNN”) de-identification tools or other rule-based tools such as the MITRE identification scrubber toolkit.

As used herein, Digital Imaging and Communications in Medicine (“DICOM”) studies may include PHI both in the metadata of the study, and also in the image itself in the form of burnt-in text or identifiable anatomical features (e.g. skull face). Metadata PHI is specified according to PHI policy and may be processed using the common PHI components described herein. Burnt-in text detection and removal and facial feature detection and removal may both be available to be called separately, allowing processing to be optimized for the presented data stream. In some implementations, DICOM images may be loaded into the de-identification pipeline from a filesystem. In some embodiments, a Picture Archive and Communication System (“PACS”) listener may be configured to receive data from a PACS server and store DICOM files to a staging area for ingestion. Additionally or alternatively, a pipeline enabled using techniques described herein may connect a PACS listener module directly to an ingestion service and bypass the staging area.

As used herein, research data export (“RDE”) waveforms are a proprietary waveform format from Philips Patient Monitors. The data consists of sets of four files, where each set contains an eight-hour segment of data from one monitor. Three of the files contain waveform metadata, including both technical details (e.g., sampling rate, channel configuration), as well as PHI such as patient MRN or dates. The fourth file is a binary file containing the raw waveforms, and is considered to not be identifiable. The monitors may be connected, for instance, to a central nurse station, which may be configured with a common internet file system (“CIFS”) mount point on which these files are saved every eight hours (or at some other periodic time interval).

Generally, in one aspect, a progressive de-de-identification method may include: receiving one or more data sets associated with one or more subjects, each of the one or more data sets containing a plurality of data points associated with a respective subject of the one or more subjects, wherein the plurality of data points include a plurality of identifying features that are usable to identify the one or more subjects; processing the one or more data sets in accordance with a first de-identification policy to generate first de-identified data, wherein the first de-identified data lacks at least one of the plurality of identifying features; transmitting the first de-identified data to a first outside entity having a first level of trust; processing the first de-identified data in accordance with a second de-identification policy to generate second de-identified data, wherein the second de-identified data lacks at least another of the plurality of identifying features; and transmitting the second de-identified data to a second outside entity having a second level of trust that is less than the first level of trust.

In various embodiments, the method may further include: processing the second de-identified data in accordance with a third de-identification policy to generate third de-identified data, wherein the third de-identified data lacks at least a third identifying feature of the plurality of identifying features; and transmitting the third de-identified data to a third outside entity having a third level of trust that is less than the second level of trust.

In another aspect, a method may include: receiving de-identified data, wherein the de-identified data includes one or more de-identified data sets associated with one or more subjects that is generated from one or more raw data sets associated with the one or more subjects, each of the one or more raw data sets containing one or more data points associated with a respective subject of the one or more subjects, wherein the one or more data points include one or more identifying features that are usable to identify the respective subject, and wherein at least some of the one or more identifying features are absent from or obfuscated in the de-identified data; determining one or more labels associated with each of the one or more de-identified data sets, wherein each of the one or more labels identifies an attribute of the respective de-identified data set; applying at least some of the one or more de-identified data sets as input across a trained machine learning model to generate one or more respective outputs, wherein each of the one or more respective outputs is indicative of whether the respective de-identified data set has the attribute; comparing the one or more outputs to the one or more labels to determine a measure of vulnerability of the de-identified data to re-identification; and based on the comparing, rejecting or accepting the de-identified data.

In various embodiments, the attribute may include a version of one or more handlers used to process the one or more raw data sets. In various embodiments, each of the one or more labels may indicate whether a date or time data point in the respective de-identified data set occurs before or after a threshold date or time. In various embodiments, the one or more de-identified data sets comprise a plurality of de-identified data sets. In various embodiments, the at least some of the plurality of de-identified data sets may include a training portion of the plurality of de-identified data sets. In various embodiments, the method may further include training the machine learning model using the training portion of the plurality of de-identified data sets. In various embodiments, the applying may include applying a remaining validation portion of the plurality of de-identified data sets as input across the trained machine learning model as validation of the training.

In various embodiments, the one or more subjects may include one or more patients, and the one or more raw data sets associated with the one or more subjects may include medical records associated with the one or more patients. In various embodiments, the trained machine learning model may include a random forest or AdaBoost component.

In addition, some implementations include one or more processors of one or more computing devices, where the one or more processors are operable to execute instructions stored in associated memory, and where the instructions are configured to cause performance of any of the aforementioned methods. Some implementations also include one or more non-transitory computer readable storage media storing computer instructions executable by one or more processors to perform any of the aforementioned methods.

Techniques, systems, and frameworks described herein give rise to a variety of technical advantages. Providing a centralized and easily modifiable system and framework for data de-identification makes it possible to de-identify new types of data, or syntactic variations of existing data, with relative ease. Additionally, techniques described herein relating to progressive de-identification may conserve considerable computing resources and/or time by avoiding duplication of de-identification efforts, all while maintaining a “need to know” environment that facilitates outside research and/or data analytics while reducing unauthorized data re-identification. As another example, techniques and frameworks described herein facilitate data linkage between disparate de-identified sets of data, such that it is possible to later reassemble the data (assuming access to a secure facility) for a variety of purposes. This is beneficial, for instance, because it allows storage of de-identified data at a less secure site, with reassembly permitted by authorized personnel for a limited set of circumstances, while also conserving storage at the secure site. In some cases the secure site (or more alternatively, the site that generated the raw data) may only store the data required for reassembly (e.g., lookup tables, reverse hash functions, date/time shifts), while the offsite storage may only store de-identified data. Consequently, neither the secure site nor the offsite storage can be infiltrated individually by a malicious party to re-identify subjects—infiltration at both sites would be required, which may prove more difficult.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles of the embodiments described herein.

FIG. 1 illustrates schematically an example environment in which selected aspects of the present disclosure may be implemented, in accordance with various embodiments.

FIG. 2 illustrates schematically an example hierarchal taxonomy that may be used in various embodiments to classify data associated with a subject.

FIG. 3 depicts an example method of practicing selected aspects of the present disclosure, in accordance with various embodiments.

FIG. 4 illustrates schematically an example computer architecture, in accordance with various embodiments.

FIG. 5 depicts another example of an environment in which selected aspects of the present disclosure may be implemented, in accordance with various embodiments.

FIG. 6 depicts another example of an environment in which selected aspects of the present disclosure, including progressive de-identification, may be implemented, in accordance with various embodiments.

FIG. 7 depicts another example of an environment in which selected aspects of the present disclosure may be implemented for secure cloud storage/processing, in accordance with various embodiments.

FIG. 8 depicts another example of an environment in which selected aspects of the present disclosure may be implemented, in accordance with various embodiments.

FIG. 9 schematically depicts one example of how load balancing may be implemented between multiple de-identification modules, in accordance with various embodiments.

FIG. 10 depicts an example method of practicing selected aspects of the present disclosure, in accordance with various embodiments.

FIG. 11 depicts an example method of practicing selected aspects of the present disclosure, in accordance with various embodiments.

### DETAILED DESCRIPTION

As data collection and proliferation becomes more and more ubiquitous, it becomes increasingly important to protect various types of protected data while also allowing the data to be leveraged to its full potential. For example, various types of data may be subjected to de-identification or anonymization processing in which data that are usable to identify an individual or group may be scrubbed while other data may be maintained in some form so that it can be used for various beneficial purposes.

Patient healthcare data can be extremely useful for a variety of purposes, such as disease research, development of drugs and other treatments, etc. However, this data is typically considered highly sensitive, and therefore may be covered by national, regional, hospital, or business regulations. Each set of regulations may impose or alter requirements for how patient healthcare data is handled. This particularly applies to de-identification, in which protected health information (“PHI”) is identified and either modified (e.g., obfuscated) or removed in order to limit risk to patients and care providers. Various agreements or regulations may identify any number elements, or may allow for the elements to be transformed rather than removed, balancing research requirements and other privacy safeguards with re-identification risk. Efforts to create a software system capable of producing de-identified output acceptable to all standards have failed. Instead, software systems have been created piecemeal that are tailored for each application.

Accordingly, the present disclosure is directed to methods and apparatus for centralized de-identification of protected data associated with subjects in multiple modalities based on a hierarchal taxonomy of policies and handlers. For example, in the healthcare context, techniques described herein may be implemented to provide a centralized platform that is capable of processing multiple micro-batched data sets, data streams, and/or sources containing multiple data types and/or data modalities. The platform may be easily configurable to perform de-identification in accordance with a variety of different regulations, as well as to facilitate other features such as deduplication, auditing, and/or discoverability. In some embodiments, the platform may make use of a hierarchal taxonomy to classify individual data points, as well as to select handlers to process the data points in accordance with their classifications. Techniques disclosed herein create a single software service to act as a single point of configuration and to perform centralized PHI de-identification for all processing modalities. A flexible configuration syntax is described that can cover HIPAA and other use cases, and be extended as needed to localized requirements. All modality-specific components make use of this central service, ensuring that the outputs are consistently de-identified to meet regulatory requirements and to facilitate creation of a multi-modal linked dataset. Data from multiple sources about the same subject may be linked longitudinally across various managed health systems, such as electronic medical records (“EMR”), electronic health records (“HER”), hospital information systems (“HIS”), and/or radiology information systems (“RIS”). This may be maintained, for instance, through multiple de-Identification passes carried out at different stages in the life-cycle of a subject.

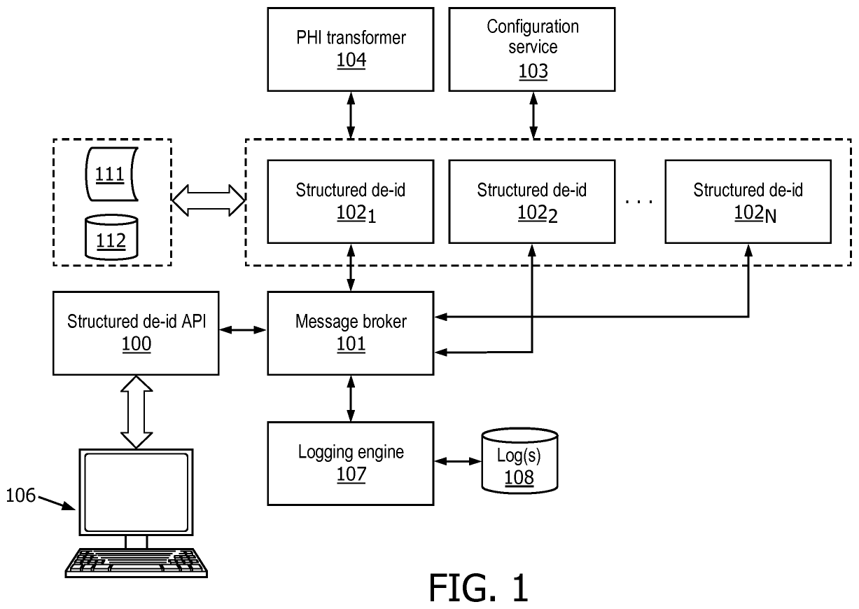

Referring to FIG. 1, an example environment in which selected aspects of the present disclosure may be implemented is depicted schematically, in accordance with some embodiments. Each of the depicted elements or modules may be implemented using any combination of hardware or software. While a particular arrangement of components is depicted in FIG. 1, this is not meant to be limiting. In various embodiments, one or more components/modules may be added, deleted, or its functionality may be distributed across one or more other components/modules. Moreover, the components depicted in FIG. 1 may be implemented across any number of computing systems and computing devices that are communicably coupled with one another over one or more computer networks.

A structured de-id application programming interface (“API”) module 100 may receive, e.g., from one or more client devices 106 operated by medical personnel, researchers, patients, etc., a request that includes or identifies a payload of data to be de-identified. The request may also be made available through events as and/or when a new dataset arrives and/or is imported into the system. This may provide a continuous de-identification pipeline for the datasets. In some implementations, the request may take the form of a Representational State Transfer call, or “REST.” REST is an architecture style for designing networked applications. More specifically, REST is a commonly-used stateless, client-server, cacheable communications protocol that is often (but not exclusively) used on top of the hypertext transfer protocol (“HTTP”). In other embodiments, other protocols such as the Common Object Request Broker Architecture (“CORBA”), remote procedure calls (“RPC”), or the Simple Object Access Protocol (“SOAP”) may be used in addition to or instead of REST.

In some implementations, the payload may specify, e.g., within external data sources 111 (e.g., remote hospitals, deployed personal physiological sensors, etc.) or internal data sources 112 (e.g., EMRs, hospital information systems, or “HIS”, etc.), input data or other data sources that provide data to be de-identified, as well as locations for storing the resulting de-identified data. Input data may come in various formats, such as coma separate values (“CSV”), relational databases, JavaScript Object Notation (“JSON”), Health Level Seven (“HL7”), DICOM, PACS, and so forth. Additionally or alternatively, in some embodiments, the payload may specify a corresponding schema file that declares the data type and the kind of de-identification required for each data element, and/or the output location where the de-identified data should be stored. The payload may be encoded using various protocols, such as JSON, extensible markup language (“XML”), and so forth.

Client device(s) 106 may include, for example, one or more of: a desktop computing device, a laptop computing device, a tablet computing device, a mobile phone computing device, a computing device of a vehicle of the user (e.g., an in-vehicle communications system, an in-vehicle entertainment system, an in-vehicle navigation system), a standalone interactive speaker, a smart appliance such as a smart television, and/or a wearable apparatus of the user that includes a computing device (e.g., a watch of the user having a computing device, glasses of the user having a computing device, a virtual or augmented reality computing device). Additional and/or alternative client computing devices may be provided.

In response the request received from the client device(s) 106, in various embodiments, structured de-id API module 100 may then send a message to a message broker 101. Message broker 101 may be a message broker software program, sometimes referred to as “message-oriented middleware,” that is configured to queue and relay various messages between various components depicted in FIG. 1. In some implementations, message broker 101 may take the form of a RabbitMQ message bus. RabbitMQ may be used to implement protocols such as the Advanced Message Queuing Protocol (“AMQP”), the Streaming Text Oriented Messaging Protocol (“STOMP”), the Message Queuing Telemetry Transport (“MQTT”), and other protocols. The message sent from structured de-id API module 100 to message broker 101 may indicate that a new de-identification job has been created (i.e. a “job creation message”). Structured de-id API module 100 may also send a job status message to message broker 101 and set the job status to be “in-queue.” In some implementations, the structured de-id API module 100 may also query message broker 101 for the status of a submitted job by sending a query message to message broker 101. Additionally or alternatively, other protocols may be employed to exchange data between components of FIG. 1, such as NiagraFiles (“NiFi”).

One or more structured de-id modules 1021N may be configured to interface with message broker 101 and listen for job creation messages that they can accept for processing. Each structured de-id module 102 may be configured to locate, based on the job creation message, the input data and the schema files (if present), and to process the input data using techniques described herein, e.g., to generate output data that is de-identified. As will be described below, in some embodiments, each structured de-identification module 102 may classify individual data points of the input data in accordance with a hierarchal taxonomy and then further process the individual data points based on the classifications. In various embodiments, each structured de-id module 102 may update its job status to ‘de-id started’ by sending another message to message broker 101.

In various embodiments, an external configuration service module 103 may be configured to supply configurations that should be used by a given structured de-identification module 102 during its de-identification and/or de-duplication processing of the input data. For example, in some embodiments, a PHI transformer module 104 may host (or otherwise proved access to) a library of handlers (e.g., software functions, remote software agents, micro-services, etc.) that each is configured to perform a particular action (e.g., de-identification, deduplication, etc.) on a particular classified data point. In some embodiments, each structured de-identification module 102 makes specific calls to PHI transformer module 104 for the de-identification of specific attributes in the data. If de-identification process succeeds, the structured de-id module 102 may send a ‘de-id complete’ or similar message to message broker 101; otherwise structured de-id module 102 may send a ‘de-id failed’ or similar message to message broker 101. Put another way, message broker 101 maintains a list (or queue) of active de-identification jobs being performed by one or more structured de-id modules 102 based on requests received at structured de-id API 100.

Configuration service module 103 may act as a single point of configuration available to users, so that users are able to customize and/or create new policies and/or handlers to deal with various types of data as needed. The configuration(s) maintained by configuration module 103 may be extensible to support different data types and/or different data modalities, and/or to adjust various handlers and aspects of handlers, such as which hash type is used, which dateshift is employed, and so forth. In some implementations, configuration service module 103 may be operable to provide centralized storage, validation, and versioning of all system configurations.

In various implementations, a logging engine 107 may listen (e.g., periodically poll) the job queue maintained by message broker 101 and record the status in one or more logs 108 (e.g., a plaintext file or postgreSQL database). Logging engine 107 may also return the status of a de-identification job if a query message is sent to message broker 101. In some embodiments, logging engine 107 may create logs 108 for a variety of purposes, such as auditing, provenance tracking, and so forth.

As noted previously, in various implementations, techniques described herein may rely on a hierarchal taxonomy to classify individual data points of input data. These classifications may be used, e.g., by structured de-id modules 102_{1-N}, to select handlers, e.g., from a library of handlers provided by PHI transformer 104. The selected handlers may then be applied to (e.g., used to process) the input data to generate de-identified and/or de-duplicated data that is usable for various purposes, such as studies, research, etc. Each handler may operate on a particular data type and/or modality. Individual data points may be obtained from a variety of sources (e.g., from 111 and/or 112), such as structured data files (e.g., JSON, CSV, etc., which may contain recorded physiological measurements, lab results, treatments applied, prescriptions, diagnoses, etc.), detected in images from DICOM or PACS data (e.g., detected within the images such as CT scans or MRI data, or within associated metadata), extracted from EMRs (which could include free-form text that describes diagnoses, treatments, prescriptions, etc.), obtained from streams of data produced by various medical equipment (e.g., heartrate sensors, weight scales, glucose meters, pulse oximeters, etc.), and so forth.

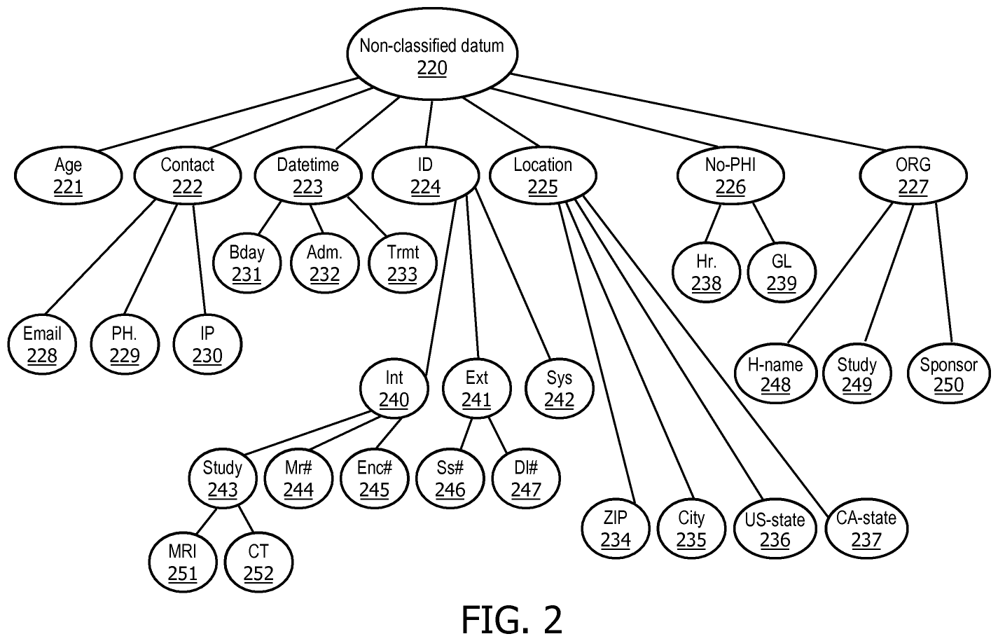

FIG. 2 schematically depicts one example of a hierarchal taxonomy that may be used in various embodiments. Starting at root node 220, a data point may be first classified with a general data type, such as age 221, contact 222, datetime 223, ID 224, location 225, no-PHI (i.e., non-protected health information) 226, and organization (ORG) 227. At least some of the data types may include a sub-taxonomy of modalities. For example, contact 222 may have sub-modalities of email 228, telephone (“PH”) 229, and IP address 230, among others. Datetime 223 may have sub-modalities of birthday 231, admission date/time 232, treatment date/time 233, and so forth.

As noted previously, ID 224 may have sub-modalities of internal 240, external 241, and system 242, among others. In some embodiments, each of these sub-modalities 240-242 may itself have a sub-taxonomy of modalities. For example, internal 240 has sub-modalities of study identification number 243, medical record number 244, and hospital encounter number 245, among others. External 241 has sub-modalities of social security number 246 and driver's license number 247, among others. And study identification number 243 has sub-modalities of MRI scan number 251 and CT scan number 252, among others.

Location 225 has a sub-taxonomy of modalities that include ZIP code 234, city 235, US-state 236, Canadian state CA-state 237, and may include various other modalities as applicable. ORG 227 includes a sub-taxonomy of modalities that includes hospital name 248, study (or trial) name 249, and study sponsor 250.

No-PHI 226 has a sub-taxonomy of modalities that includes, for instance, physiological parameters and other data points that are not usable (at least alone) to identify a subject. In FIG. 2, for instance, no-PHI 226 includes heartrate 238 and glucose level 239. These are not meant to be limiting, and any other physiological parameter may be included in a hierarchal taxonomy as described herein. Indeed, techniques described herein allow for the handling of a wide variety of physiological data, such as structured data received from physiological sensors (which may be organized, for instance, in JSON) and other types of data, such data formatted in the DICOM or PACS standards.

Data points or streams of data that are to be processed using techniques described herein may be classified using a hierarchal taxonomy such as that depicted in FIG. 2. In some embodiments, an initial set of PHI data types (e.g., 221-227) is defined as a starting point, to cover a majority of use cases, and to serve as a basis for additional customization. The hierarchal taxonomy may be configured to define increasing levels of detail of PHI data type (i.e., sub-taxonomies of modalities for each data type as described above).

In some embodiments, each data point (or data points) may be classified or “tagged” with a PHI classification that includes a full path in the hierarchal taxonomy, which in examples described herein are separated by colons (‘:’) though this is not meant to be limiting. For example, an MRI identifier for a particular study may be classified or tagged as “id:int:study-id:mri.” A CT identifier for a particular study may be classified or tagged as “id:int:study-id:ct.” A United States Veterans Affairs account number may be classified or tagged as “id:ext:account-no:us-va.” And so on.

The data classifications determined using a hierarchal taxonomy such as that depicted in FIG. 2 may be used, along with their corresponding policies, to determine how each data point of input is handled (e.g., de-identified, unaltered or passed through, dropped, etc.). In some embodiments, a classification of a data point (or a set or stream of data points sharing a type/modality) may be associated with a particular policy. The policy may identify a handler to be used, e.g., by one or more structured de-id modules 102_{1-N}, to process the data point(s). In other words, policies are defined using the hierarchal taxonomy. In some embodiments, general PHI classes (e.g. ‘id’) may have a fail-safe policy, and more specific or granular classes (e.g. ‘id:int:mrn’) may be granted a more permissive policy that includes a handler that does something other than drop the data point (e.g., obfuscate, shift, mask, etc.) as required to allow research.

In some embodiments, the most specific or granular applicable policy may be applied for each tag handled by the PHI transformer 104 (see FIG. 1). Suppose an incoming data element was tagged “id:int:mrn.” That may match the policy for ID 224 (see FIG. 2) which may map to a “drop” handler (e.g., delete or remove data), and it may also match the policy for “id:int:mrn” (243 in FIG. 2), which may be “lookup-table.” In this case, “id:int:mrn” (243) is the more specific or granular policy and therefore a different handler would be applied to the data point. In some implementations, the default policy handler for a high-level classification of potential PHI may be “drop.” If a given data point does not match any more specific classification in the policy then the data point may be redacted and/or replaced with a label such as “removed”.

In some embodiments, PHI policies may be defined using the JSON format. The following is one non-limiting example:

```
{
  ‘datetime’: ‘datetime-global-shift’,
  ‘id’: ‘drop’,
  ‘id:int’: ‘lookup-table’,
  ‘id:sys:row-id’: ‘passthrough’,
  ‘location’: ‘drop’
}
```

This policy indicates that all data points classified as datetimes will be transformed using a “datetime-global-shift” handler. Data points classified as identifiers (“id”) will be dropped by default, however internal (“id:int”) identifiers will be mapped to handlers, e.g., by PHI transformer 104, using a lookup table. Data points classified as “id:sys:row-id” (e.g., database row ids) will be allowed through unmodified (“passthrough”). Data points classified as locations (e.g., ZIP codes, cities, states, etc.) will be dropped.

As noted previously, a library of handlers may be maintained, e.g., by PHI transformer 104. In some embodiments there may be a variety of default PHI handlers available to handle the majority of cases. The following sub-sections list non-limiting examples of policies, each including a policy name (in quotes), description, and input, output, and configuration options of each policy handler.

‘Age-Handler-Basic’: Basic Processing of Patient Age

In accordance with HIPAA and other policies, patient ages of 90 or above may be considered special PHI, e.g., due to the scarcity of such patients. This handler considers tagged elements as ages—values below a threshold are passed through unmodified. Ages equal to and/or above the threshold are substituted for the configured value.

Input: Numeric age, years

Configuration Options:

Threshold for cutoff, years

Replacement value, numeric, default ‘130’—allows comparison operators to work as expected (‘greater than’, ‘less than’), large enough to be apparent as artificial, while still being near to physiological possibility.

Output: Numeric age, years

### ‘Datetime-Global-Shift’: Global Datetime Shift

This is the default handler for datetime values in some embodiments. It applies a global shift to all data points having the data type of datetime. In some implementations, datetime input are expected to comply with ISO 8601, including source time zone. The default output may be converted, for instance, to Greenwich Mean Time (“GMT”), which eliminates possibility of location leakage through time zone information, or date leakage through daylight savings time information. In some embodiments, the date shift is specified in days, as a shift of years can result in nonsensical dates due to leap years (e.g. Feb. 29, 2043).

Input: Datetime in ISO 8601 format. If no time zone is specified, offset of +0 may be assumed.

Configuration options for this handler may include:

Number of days to shift the output

Output time zone (default ‘GMT’, 0 time offset)

Output: Datetime using ISO 8601 standard, including time zone

### ‘Drop’: Removal of Original Value

Element is removed, replaced with the value “<removed>”.

### ‘Hash’: Hashing Function

This handler passes the data point through one or more defined hashing functions.

Input: Any data element

The following are non-limiting configuration options:

Hash level (‘Low’, ‘Medium’, or ‘High’): security level of hash function, could map to, for instance, md5, ssh512, and pbkdf2_hmac, although other mappings are possible.

Salt: Salt of hash function, kept secret from all downstream processes. Salt is random data that is used as additional input to a one-way hash function. Salts are beneficial because, for instance, they defend against attacks such as dictionary attacks and/or pre-computed rainbow table attacks.

Output: Hashed output

### ‘Lookup-Table’: Dynamic Lookup Table Creation and Value Replacement

The data point is referenced against a defined lookup table, which may be segregated according to the complete PHI hierarchal taxonomy. If an existing element is discovered then the existing lookup value is returned, otherwise a new universally unique identifier (UUID) is generated, added to the lookup table, and returned.

As an example, if MRN 55 and encounter ID 55 both exist and are labeled by general PHI classification as ‘id’, they will both receive the same UUID conversion. However, if they are properly classed by subtypes as ‘id:int:mm’ and ‘id:innencounter-id’, they will be sorted in separate lookup tables and be assigned unique identifiers.

Input: Any data element

Output: UUID

### ‘Passthrough’: Pass-Through of Original Value

Data element is unmodified and returned in original form.

Additionally or alternatively, in some embodiments there may be an expandable library of special PHI handlers that may be required by various sites or localities. The following sub-sections list non-limiting examples of such policies, with the policy name in quotes, description, and input, output, and configuration options of each transformation.

### ‘Age-Handler-Advanced’: Advanced Age Handling with Multi-Resolution De-Identification

This handler builds on the basic age handler with the implementation of age resolution reduction. Various policies or regulations may require that patients of different ages have varying levels of resolution retained in their ages. For example, it may be necessary for neonatal intensive care unit (“NICU”) algorithm development that the patient's age is available at a day resolution, whereas older patients' ages may be limited to year resolution, or binned into 2, 5, or 10 year increments, depending on the potential numbers of patients in those age ranges.

This handler allows definition of age cutoffs and resolutions, where cutoffs and resolutions are specified as pairs (cutoff₁, resolution₁), (cutoff₂, resolution₂), . . . (cutoffₙ, resolutionₙ), where ages from 0 to cutoff₁ (days) are down-sampled to resolution₁, ages from cutoff₁ to cutoff₂ are down-sampled to resolution₂, etc., and ages above cutoffₙ are replaced with an old age replacement value. Default values replicate the functionality of the basic age handler.

Input: Age in years, which may be an integer greater than or equal to zero in some embodiments.

Configuration options are:

List of (age-cutoff, resolution): age-cutoff (days) specifies a boundary age, resolution (days) specifies resolution of down-sampling. Default is (32850, 365), which corresponds to a 90 year threshold, with ages less than 90 being down-sampled to 1-year increments.

Old Age Replacement Value: 47450 (130 years in days), same default as basic age handler, or some other value.

Output: Age in years, which may be an integer greater than or equal to zero in some embodiments.

### ‘Birthday’: Date of Birth Handling

Ages greater than ninety may be considered PHI according to HIPAA and other policies, along with any other information that could be used to derive age, e.g. date of birth. The result of this policy is that dates that are birthdates may not simply be shifted, but must first be used to calculate the person's age relative to some recent baseline date (e.g. date of hospital admission), evaluated relative to the PHI cutoff threshold (e.g., ninety years), and either shifted, shifted with resolution reduction, or replaced. Ages may be calculated from the input reference datetime to the birthday. Calculated ages may be processed with the ‘age’ handler as defined in the policy. If none is present, this handler may default to the ‘age-handler-basic’. Ages may then be subtracted from the reference time, and may be shifted using the ‘datetime’ policy. If ‘datetime’ policy is contextual date shift, required elements may be passed to this function as well.

Input: Date of birth, reference date, additional contextual data if ‘contextual-datetime-shift’ is selected. ISO 8601 format

Output: Date, ISO 8601 format

### ‘Contextual-Datetime-Shift’: Contextual Datetime Shift

For wider release de-identification scenarios (e.g. creation of public datasets), a global date shift may be considered insufficient to mitigate re-identification risk, as any single patient's data may be used to discover the shift for all patients. In these instances a contextual date shift may be used. Every patient may have a different (e.g., unique) dateshift, or every ICU encounter, every hospital encounter, etc. With this handler, all events from the same context (e.g. hospital encounter) may receive the same dateshift and may be chronologically ordered relative to one another, but events from different contexts may receive different date shifts. In some implementations, for a given context, a random date shift may be chosen between the specified minimum and maximum shifts. Day of week and seasonality are optionally preserved.

Input: Datetime in ISO 8601 format. If no time zone is specified, offset of +0 is assumed.

Context: list of (phi-type, phi-value), as required by configuration

Here are some example configuration options:

Minimum date shift (days), default 50 years (18250 days)

Maximum date shift (days), default 75 years (27375 days)

Output time zone (default ‘GMT’, 0 time offset)

Preserve day-of-week (Boolean), default True

Preserve season (Boolean), default True

Required context: list of phi-type, e.g. [′id:int:mrn′, ‘id:int:encounter-id’] for per-encounter time shift

Output: Datetime in ISO 8601 format, including time zone

### ‘Defined-Lookup-Table’: Defined Lookup Table Replacement

This handler may use lookup tables to substitute values (e.g. city or hospital names) with human-friendly names.

Input: string or numeric value

Configuration Options:

Set of lookup tables, dictionary with keys of phi-type, and values as another dictionary specifying the key-value pairs of the lookup table.

Output: Mapped return value. If lookup table is not found, return value is ‘<table-not-found>’. If table is found but value is not found, return value is ‘<lookup-key-not-found>’.

### ‘Lookup-Table-Formatted’: Value Replacement with a Formatted Random Value

Many identifiers are given in a characteristic format, and some systems that expect these formats can break if arbitrary UUIDs or random values are presented. Examples include US social security numbers (### ## ####) and US phone numbers ((###) ###-####)).

For each phi-type a lookup table is generated, and random strings are generated according to the defined pattern until a unique string is found, up to 10 attempts before failure.

In some embodiments, if the input expression does not have sufficient space for entropy it may become impossible to randomly assign new values. For example, the pattern ‘[0-9]’ will generate a single digit 0-9, but can only create 10 total unique values, and will fail if an 11th is requested.

Input: value

Configuration Options:

Set of formats, dictionary with keys of phi-type, values as regular expressions (see https://github.com/crdoconnor/xeger for examples).

Output: formatted replacement value. Value ‘<insufficient-entropy>’ if values cannot be discovered.

### ‘Fixed-Value’: Fixed Value Replacement

Returns constant value as defined

Input: value

Configuration options: Dictionary with keys of phi-type, values as fixed replacement value

Output: Replacement value

### ‘Regex-Replace’: Masking

Replace input via regular expression. E.g. can be used to retain a US telephone area code of a phone number with following pattern: ‘\ ((\ d{3}) \) \ d{3}-\ d{4}’ ‘\ 1 xxx-xxxx’, which will replace ‘(123) 456-7890’ with ‘(123) xxx-xxxx’.

Input: string

Configuration options: Dictionary with keys of phi-type, values as search and replace regex.

Output: modified string

### ‘us-location’:United States Location Processing

Implementation of HIPAA rules on US location processing of zip codes

Input: US zip code

Output: HIPAA-compliant US zip code

### ‘Value-Noise’: Noising

This policy handler may be used to add numeric noise to input, to prevent any patient from matching actual data completely. This is intended to prevent an attacker with knowledge of a single patient from identifying that patient in the dataset.

Input: numeric value

Configuration options: Dictionary with keys of phi-type, variance of Gaussian noise distribution to sample noise factor

Output: N(input, \ sigma²)

The following table contains a non-limiting example of input data that may be identified in a payload of a request received at structured de-id API 100, and which is to be processed (e.g., de-identified) by components such as one or more structured de-id modules 1021N. Each row of the table corresponds to a particular medical event, but this is not intended to be limiting, and input data may take other forms.

EXAMPLE INPUT TO BE DE-IDENTIFIED

{″RESULT_VAL″: ″″, ″RESULT_UNITS″: ″″, ″SNOMED_CODE″:
″43173001″, ″Deid_MRN″: ″10013″, ″PERFORMED_DT_TM″:
″2001-06-16T18:03:00″, ″EVENT″: ″Orientation″}
{″RESULT_VAL″: ″″, ″RESULT_UNITS″: ″″, ″SNOMED_CODE″:
″43173001″, ″Deid_MRN″: ″10013″, ″PERFORMED_DT_TM″:
″2001-06-18T11:57:00″, ″EVENT″: ″Orientation″}
{″RESULT_VAL″: ″1.12″, ″RESULT_UNITS″: ″mg/dL″, ″SNOMED_
CODE″: ″70901006″, ″Deid_MRN″: ″10013″, ″PERFORMED_DT_
TM″: ″2001-06-18T13:42:00″, ″EVENT″: ″Creatinine″}
{″RESULT_VAL″: ″36.8″, ″RESULT_UNITS″: ″DegC″, ″SNOMED_
CODE″: ″123979008″, ″Deid_MRN″: ″10013″, ″PERFORMED_DT_
TM″: ″2001-06-17T21:26:00″, ″EVENT″: ″Temp C″}
{″RESULT_VAL″: ″88″, ″RESULT_UNITS″: ″kg″, ″SNOMED_CODE″:
″225171007″, ″Deid_MRN″: ″10013″, ″PERFORMED_DT_TM″:
″2001-06-15T20:13:00″, ″EVENT″: ″Weight (kg)″}

The following table contains a non-limiting example of a corresponding input schema which also may be identified in a payload of a request received at structured de-id API 100. Each row specifies how a particular type of data in the input data above should be handled (e.g., de-identified, dropped, etc.).

EXAMPLE INPUT SCHEMA

```
{
  ″path″: ″$.Deid_MRN″,
  ″datatype″: ″string″,
  ″PHIClass″: ″patientID″,
  ″description″: ″″
}
{
  ″path″: ″$.PERFORMED_DT_TM″,
  ″datatype″: ″datetime″,
  ″PHIClass″: ″datetime″,
  ″description″: ″″
}
{
  ″path″: ″$.SNOMED_CODE″,
  ″datatype″: ″string″,
  ″PHIClass″: ″NonPHI″,
  ″description″: ″″
}
{
  ″path″: ″$.EVENT″,
  ″datatype″: ″string″,
  ″PHIClass″: ″NonPHI″,
  ″description″: ″″
}
{
  ″path″: ″$.RESULT_VAL″,
  ″datatype″: ″numeric″,
  ″PHIClass″: ″NonPHI″,
  ″description″: ″″
}
{
  ″path″: ″$.RESULT_UNITS″,
  ″datatype″: ″string″,
  ″PHIClass″: ″NonPHI:Enumerated″,
  ″description″: ″″
}
```

The first field of the sample input schema set forth above is a string having the path “$.Deid_MRN,” wherein “$” may represent a path variable and “MRN” stands for “medical record number,” and which is labeled under “Deid_MRN” in the example input data above. The first field has a class of “patientID,” which may be PHI and therefore may be processed using a “patientID” handler to obfuscate the patient's identity. The second field has a type of “PERFORMED_DT_TM” and specifies that the datetime at which the event occurs should be handled using the “datetime” handler, which may, for instance, shift or otherwise obfuscate the date. The last four entries of the schema specify handlers for non-protected health information (“NonPHI” or “noPHI” elsewhere herein), and include, from top to bottom, a SNOMED_CODE code that identifies the medical event, an event (“EVENT” in the input data above), a RESULT value (e.g., numeric), and RESULT_UNITS (e.g., kg, ml, etc.)

The following table contains an example of de-identified output of the sample input data set forth above, as it might appear after processing using techniques described herein using the input schema set forth above.

EXAMPLE DE-IDENTIFIED OUTPUT

{u′RESULT_VAL′: ′REMOVED:INVALID NUMBER′, u′RESULT_
UNITS′: u′′, u′SNOMED_CODE′: u′43173001′, u′Deid_MRN′:
u′MRN:c25b3c94bafec0f972729bc163b258a8′, u′PERFORMED_DT_
TM′: datetime.datetime(2041, 6, 29, 18, 3, tzinfo=tzutc( )),
u′EVENT′: u′Orientation′}
{u′RESULT_VAL′: ′REMOVED:INVALID NUMBER′, u′RESULT_
UNITS′: u′′, u′SNOMED_CODE′: u′43173001′, u′Deid_MRN′:
u′MRN:c25b3c94bafec0f972729bc163b258a8′, u′PERFORMED_DT_
TM′: datetime.datetime(2041, 7, 1, 11, 57, tzinfo=tzutc( )),
u′EVENT′: u′Orientation′}
{u′RESULT_VAL′: u′1.12′, u′RESULT_UNITS′: u′mg/dL′,
u′SNOMED_CODE′: u′70901006′, u′Deid_MRN′:
u′MRN:c25b3c94bafec0f972729bc163b258a8′, u′PERFORMED_DT_
TM′: datetime.datetime(2041, 7, 1, 13, 42, tzinfo=tzutc( )),
u′EVENT′: u′Creatinine′}
{u′RESULT_VAL′: u′36.8′, u′RESULT_UNITS′: u′DegC′,
u′SNOMED_CODE′: u′123979008′, u′Deid_MRN′:
u′MRN:c25b3c94bafec0f972729bc163b258a8′, u′PERFORMED_DT_
TM′: datetime.datetime(2041, 6, 30, 21, 26, tzinfo=tzutc( )),
u′EVENT′: u′Temp C′}
{u′RESULT_VAL′: u′88′, u′RESULT_UNITS′: u′kg′, u′SNOMED_
CODE′: u′225171007′, u′Deid_MRN′:
u′MRN:c25b3c94bafec0f972729bc163b258a8′, u′PERFORMED_DT_
TM′: datetime.datetime(2041, 6, 28, 20, 13, tzinfo=tzutc( )),
u′EVENT′: u′Weight (kg)′}

It can be seen in these results that the patient's medical record number, which originally was “10013,” has been transformed into a unique identifier, “c25b3c94bafec0f972729bc163b258a8.” Likewise, the input data in the field “PERFORMED_DT_TM” has been transformed. For example, the input datetime “2001-06-16T18:03:00” in the first input entry has been transformed (e.g., de-identified) into “datetime.datetime(2041, 6, 29, 18, 3, tzinfo=tzutc( )”, which in relevant part indicates the date as being in the year 2041. Similarly, the “PERFORMED_DT_TIME” in the second input entry has been transformed from “2001-06-18T11:57:00” to “datetime.datetime(2041, 7, 1, 11, 57, tzinfo=tzutc( )”, which in relevant part indicates the date as once again being in the year 2041.

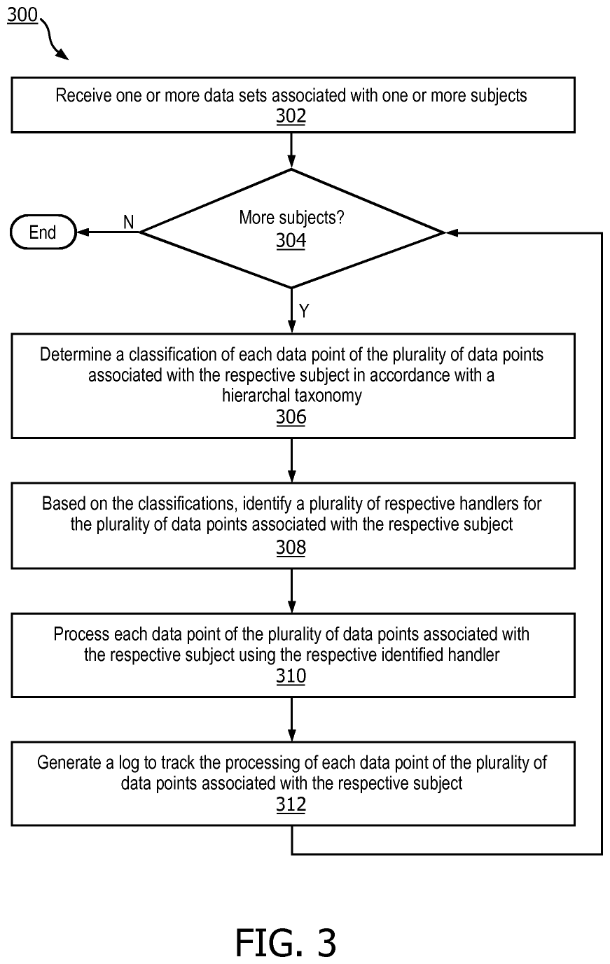

FIG. 3 depicts an example method 300 for practicing selected aspects of the present disclosure, in accordance with various embodiments. For convenience, the operations of the flow chart are described with reference to a system that performs the operations. This system may include various components of various computer systems, including components depicted in FIG. 1. Moreover, while operations of method 300 are shown in a particular order, this is not meant to be limiting. One or more operations may be reordered, omitted or added.

At block 302, the system may receive one or more data sets (e.g., identified in the payload received by structured de-id API 100) associated with one or more subjects, such as one or more patients (although this is not required). Each of the one or more data sets may contain a plurality of data points associated with a respective subject of the one or more subjects. For example, a data set may include data about multiple events associated with a single patient (e.g., as set forth in the example input data above) or events associated with multiple patients. At least some of the plurality of data points associated with the respective subject may be usable, e.g., by malicious parties, to identify the respective subject. Additionally, the plurality of data points associated with the respective subject may include multiple data types, such as those depicted in FIG. 2 (e.g., 221-227).

At block 304, a loop may begin to process the data for each respective subject of the one or more subjects, and a determination may be made whether there is additional data, and to select data for a given patient if one is available. At block 306, the system may determine a classification of each data point of the plurality of data points associated with the respective subject in accordance with a hierarchal taxonomy. As discussed previously, the hierarchal taxonomy may define, for each respective data type of the multiple data types, a sub-taxonomy of modalities (e.g., 228-249) associated with the respective data type.

At block 308, the system may, based on the classifications, identify a plurality of respective handlers for the plurality of data points associated with the respective subject. In various embodiments, at least one of the handlers may be configured to de-identify, e.g., obfuscate or drop, a data point of the plurality of data points associated with the respective subject. In some embodiments the operations of block 308 may be performed in whole or in part by PHI transformer 104, e.g., based on configuration information supplied by configuration service module 103.

At block 310, the system, e.g., by way of one or more structured de-id modules 1021N, may process each data point of the plurality of data points associated with the respective subject using the respective identified handler. The operation(s) of block 310 may, in effect, de-identify the plurality of data points associated with the respective subject. Once the data points are de-identified, they may be used for a variety of purposes, such as research, clinical trials, and so forth, without risking nefarious parties being able to identify individual subjects based on the de-identified data.

At block 312, the system, e.g., by way of logging engine 107, may generate a log to track the processing (block 310) of each data point of the plurality of data points associated with the respective subject. For example, a log may be created as a file or in database (e.g., 108 in FIG. 1) that indicates aspects of the processing such as what de-identification operations were performed, which handlers were used, which classifications applied, and so forth. In some implementations, the log may be auditable so that the processing can be reversed, effectively “re-identifying” the plurality of data points. For example, the log may include a two-way mapping between a subject's identifier (e.g., social security number, driver's license number, etc.) and a unique identifier generated therefrom. Additionally or alternatively, the log may include indications of what sort of datetime shifts were applied to input data of data type datetime. This is particularly beneficial when a different contextual date/time-shift is applied to each point of data. Of course, under such circumstances the log and/or logging engine may be protected, e.g., hosted within a secure site or system that is inaccessible to and/or protected from unauthorized parties.

Although examples described herein have primarily been focused on de-identification of healthcare-related data, e.g., for studies, trials, research, etc., this is not meant to be limiting. Techniques described herein may be applicable in a variety of other contexts in which it is desirable to de-identify data. For example, techniques and the platform described herein may be employed to de-identify data that is being transmitted from a secure site to a less secure site. Likewise, techniques described herein may be used to roll back the de-identification (re-identification) when data is returned from the less secure site back to the secure site. Moreover, techniques described herein are applicable across a variety of domains in addition to healthcare, such as finance, consumer data, or other domains in which de-identified protected data can be used for various purposes.

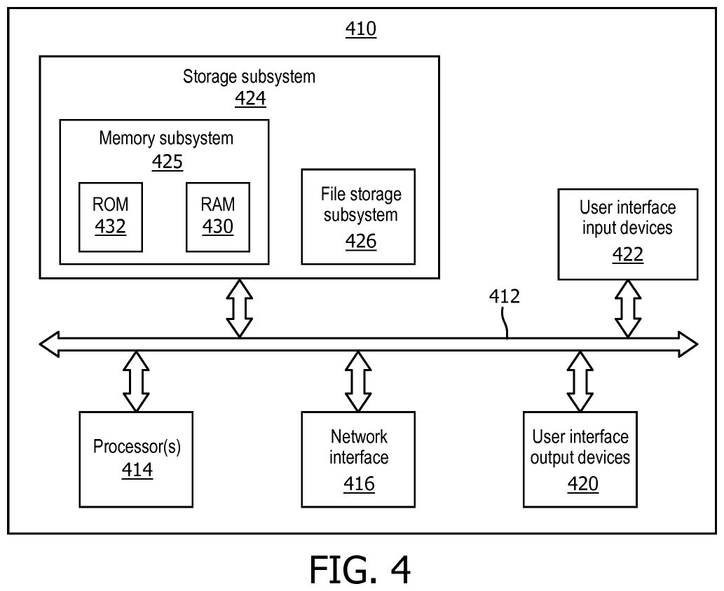

FIG. 4 is a block diagram of an example computing device 410 that may optionally be utilized to perform one or more aspects of techniques described herein. Computing device 410 typically includes at least one processor 414 which communicates with a number of peripheral devices via bus subsystem 412. These peripheral devices may include a storage subsystem 424, including, for example, a memory subsystem 425 and a file storage subsystem 426, user interface output devices 420, user interface input devices 422, and a network interface subsystem 416. The input and output devices allow user interaction with computing device 410. Network interface subsystem 416 provides an interface to outside networks and is coupled to corresponding interface devices in other computing devices.

User interface input devices 422 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computing device 410 or onto a communication network.

User interface output devices 420 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computing device 410 to the user or to another machine or computing device.

Storage subsystem 424 stores programming and data constructs that provide the functionality of some or all of the modules described herein. For example, the storage subsystem 424 may include the logic to perform selected aspects of the method of FIG. 3, as well as to implement various components depicted in FIG. 1.

These software modules are generally executed by processor 414 alone or in combination with other processors. Memory 425 used in the storage subsystem 424 can include a number of memories including a main random access memory (RAM) 430 for storage of instructions and data during program execution and a read only memory (ROM) 432 in which fixed instructions are stored. A file storage subsystem 426 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 426 in the storage subsystem 424, or in other machines accessible by the processor(s) 414.

Bus subsystem 412 provides a mechanism for letting the various components and subsystems of computing device 410 communicate with each other as intended. Although bus subsystem 412 is shown schematically as a single bus, alternative implementations of the bus subsystem may use multiple busses.

Computing device 410 can be of varying types including a workstation, server, computing cluster, blade server, server farm, or any other data processing system or computing device. Due to the ever-changing nature of computers and networks, the description of computing device 410 depicted in FIG. 4 is intended only as a specific example for purposes of illustrating some implementations. Many other configurations of computing device 410 are possible having more or fewer components than the computing device depicted in FIG. 4.

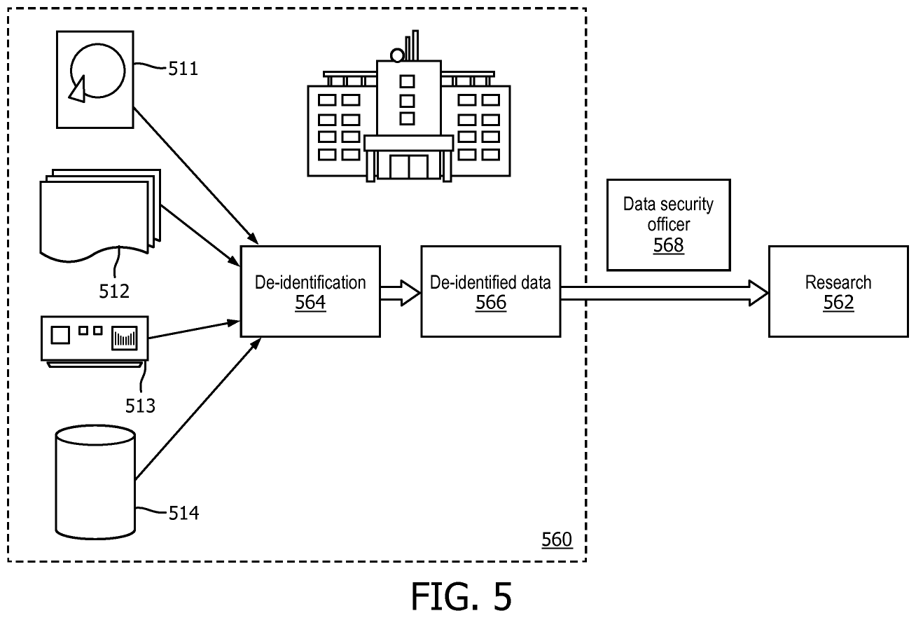

FIG. 5 depicts an example use case in which data captured and/or obtained in a secure environment—a hospital 560 in FIG. 5—is de-identified using various techniques described herein so that it can be provided to a research entity 562. In FIG. 5, a variety of data sources are available from which data can be obtained, including one or more computer hard drives 511 from which data can be extracted, for instance, from files, etc. Also present are one or more external documents 512 (e.g., documents from outside entities), one or more medical devices 513 (e.g., streams of physiological data measured from patient(s)), and one or more databases 512 (e.g., HER, EMR, HIS, which may provide access to e.g., PACS, DICOM, HL7, etc.).

Sets of data received from the various sources 511-514 may range from 100 MB CSV files and dozens of DICOM images, to 100 GB parquet files and tens of thousands of DICOM studies. Additionally or alternatively, sites that produce large amounts of data, such as large hospitals or entire healthcare systems, may have streaming capabilities to connect de-identification module 564 directly to data sources such as PACS servers or HL7 busses. In various embodiments, storage across the various data sources, such as database 514, may be flexibly configured, allowing for filesystem storage (e.g. shared filesystem via NFS, Gluster, CephFS, or other, made available via Docker data volumes). Additionally or alternatively, object storage, which is prevalent for cloud deployment (e.g. S3), may be employed. Some embodiments may employ technologies such as Ceph, Swift, NetApp, etc., though these are not meant to be limiting.

A de-identification module 564 may include one or more components depicted in FIG. 1 and described previously, and may be configured to obtain different types and/or modalities of data from the data sources 511-514 and process them (e.g., de-identify, de-duplicate, log, create reports, etc.). The output of de-identification module 564 takes the form of de-identified data 566. This de-identified data 566 may be provided to research entity 562 after being verified/inspected by one or more data security officers 568, which in some cases may be human, and/or may include software executing on one or more computing systems. In some implementations, data security officer 568 may include one or more machine learning models that are trained to receive de-identified data 566 as input and generate output indicative of how vulnerable the de-identified data 566 is to re-identification by an unauthorized party. A non-limiting example of such a machine learning model will be described shortly.

In some embodiments, multiple types of data obtained from the various sources 511-514 may be linked together such that the same patient's data can be correlated between different streams. Suppose a patient named “John Smith” has an MRN 555 and also has both DICOM and EMR data. De-identification module 564 may assign the same pseudo-identifiers (e.g., randomly-generated unique identifiers) and use the same timeshift for both EMR and DICOM data. Thus, if this patient were assigned the pseudo-identifier MRN 7371637 in an EMR pipeline, DICOM images for this patient may also be tagged with MRN 7371637.

In various embodiments, re-identification is facilitated in whole or in part by tracking provenance (e.g., the origin of each data point is preserved). In various embodiments, a source of data and/or a version number of various processes that process the data may be preserved, e.g., in a log such as log 108 in FIG. 1.

As mentioned previously, in some implementations, data security officer 568 may include one or more computing systems that are configured to apply one or more trained machine learning models to de-identified data 566 to determine how vulnerable the data is to re-identification. This is also referred to herein as identification of data “leakage.” If it is determined, based on the output of these models, that re-identification would be feasible (e.g., a risk measure satisfies a threshold, leakage is possible or even probable), an alarm may be raised to appropriate personnel that additional configuration may be warranted.

In one implementation, data security officer 568 may analyze de-identified data 566 for detectable patterns or other identifiable features in date shifts applied to data having the date type (e.g., hospital encounters, treatment dates, birthdates, etc.). In some such embodiments, each piece of de-identified data may be tagged with a label that indicates whether it came from a software configuration and/or version being tested or not. A classifier such as a Random Forest, AdaBoost, or other, may be trained to classify data as having come from the same configuration/version. Some of the data, say, 70%, may be used training, and the remainder of the data may be used for validation data. If, after training, the classifier is able to correctly predict the configuration origin of at least a threshold amount of the validation data (e.g. area under the curve, or “AUC”=0.80), then the de-identified data 566 may be considered tainted and appropriate personnel may be notified. In some implementations, the features or parameters found most important or particularly influential by the classification algorithm may be output for inspection, and may be removed or altered as needed.

Additionally or alternatively, in some embodiments, the actual dates of each data point may be used to detect leakage. For example, individual data points (e.g. rows, images, or other modality) may be tagged with their original date, or timestamp, before being date or time shifted. A threshold date may be selected, e.g. towards the beginning of the start of data collection. Each data point may be labelled with, for instance, a zero or one to indicate whether it occurred before or after the threshold. Similar to the previous example, a machine learning classifier may be trained to predict whether a given data point occurred before or after the threshold. If the classifier's performance exceeds some security threshold, the data may be considered potentially tainted as described above. In some implementations, if the classifier performs poorly, the threshold date may be advanced into the future and the process repeated.

Another aspect of the present disclosure is directed to progressive de-identification to support multiple user classes, e.g., to avoid repeated data extraction and processing as described above. In many localities and scenarios, multiple levels of de-identification are permitted or required. In the United States, HIPAA regulations allow for either “Safe Harbor” de-identification or “Expert Determination” de-identification. Safe Harbor is the simplest and safest form of de-identification, stipulating a fixed set of eighteen rules to be followed for de-identification. While this offers the greatest legal protection, it requires data to be removed that may be essential, or at least beneficial, for some types of research. As an example, Safe Harbor requires that all elements of dates be removed. This limits the ability of researchers or other interested parties to determine which patients were enrolled simultaneously—an important element for a variety of studies, including studies on the impact of intensive care unit (“ICU”) overload conditions on patient outcome.

Expert Determination may be used instead of Safe Harbor when, for instance, data is being released from a secure site/system to a less secure site (e.g., as depicted in FIG. 5) under a business-to-business agreement, such as a master research agreement. Safeguards around data storage, user access controls, contractual protections, and other factors may be considered in balancing risk and determining how processing occurs to preserve data for research while protecting patient privacy.

Typically, for a specific use case (e.g., a specific study, research, etc.), data is de-identified as aggressively as possible to allow that particular use case to be beneficial while still satisfying applicable regulations or agreements. A downside of this approach is that the de-identified data may have limited benefit outside of the specific use case. Consequently, if future use cases call for additional research on the same raw data, the raw data must often be re-extracted and re-processed because data required for the new use case has often been removed from the previously-de-identified data. As noted elsewhere herein, data sets, particular those relating to healthcare, are growing in size to terabytes and even petabytes. Accordingly, the computing and time costs associated with this re-extraction and re-processing are high.

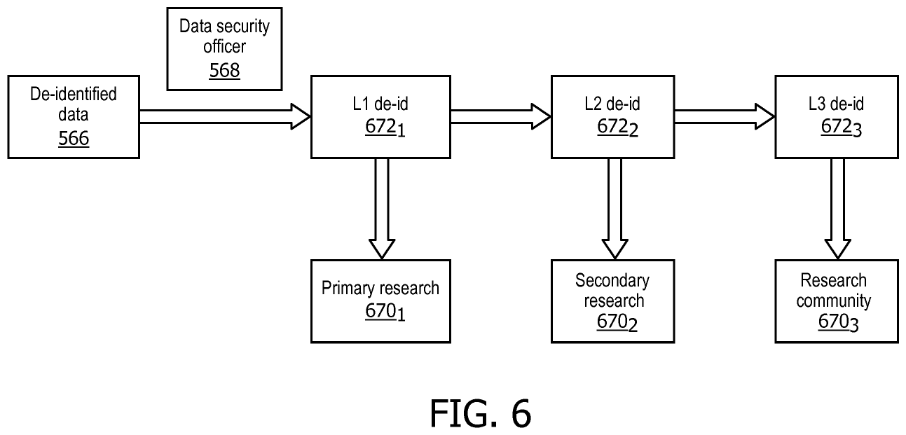

Accordingly, to address these issues, in various embodiments, what will be referred to herein as “progressive de-identification” may be implemented. Referring now to FIG. 6, the de-identified data 566 and data security officer 568 depicted in FIG. 5 are illustrated as part of a downstream pipeline that facilitates progressive de-identification. A plurality of research entities 670₁₋₃ are depicted as downstream recipients of the de-identified data 566. While three research entities 670₁₋₃ are depicted in FIG. 6, this is not meant to be limiting, and any number of downstream research entities may be serviced using techniques described herein.

Each research entity 670 may operate in accordance with different regulations or agreements. For example, first research entity 670₁ might operate under the constraints imposed by HIPAA. Second research entity 670₂ might operate under different regulations, e.g., imposed by a government or agency outside of the United States. Third research entity 670₃ might operate under a master research agreement between it and, for instance, hospital 560 of FIG. 5. Of course these are just examples, and other permutations are possible. At any rate, it may be the case that the de-identification requirements imposed on first research entity 670₁ are the least restrictive (e.g., it is a relatively highly trusted entity such as a government agency for which a strength of its security measures are known), the de-identification requirements imposed on second research entity 670₂ are more restrictive, and the de-identification requirements imposed on third research entity 670₃ are the most restrictive, e.g., because it is a private or commercial entity for which a strength of its security measures is unknown.

The progressive de-identification pipeline of FIG. 5 allows for multiple PHI policies to be applied to the data to allow for different levels of de-identification for each of the research entities 670. One policy might be applicable to extract data, e.g., from hospital 560 to a secure quarantine environment, e.g. controlled by data security officer 568. Other policies might further limit data as required by each different research entity 670. For example, a first level of de-identified data 672₁ may be provided to first research entity 670₁. The first level of de-identified data 672₁ may itself be further processed (rather than processing the original raw data), e.g., by one or more de-identification modules 102, to generate second level de-identified data 672₂ that is provided to second research entity 670₂. Similarly, the second level of de-identified data 672₂ may itself be further processed, e.g., by one or more de-identification modules 102, to generate third level de-identified data 672₃ that is provided to third research entity 670₃.

This may accelerate research by making data available more quickly for researchers, e.g., because data that is de-identified across all levels need not be “re-de-identified” for each entity. Rather, data that is already de-identified can, in some cases, simply be passed through to the next level of processing. This may be particularly advantageous when the amount of data is large and/or the processing involved with de-identification (which could include, for instance, extracting data from DICOM/PACS) is computationally expensive. This progressive de-identification technique may also increase security by facilitating a “need-to-know” environment in which researchers only have access to data elements necessary for their particular investigations.

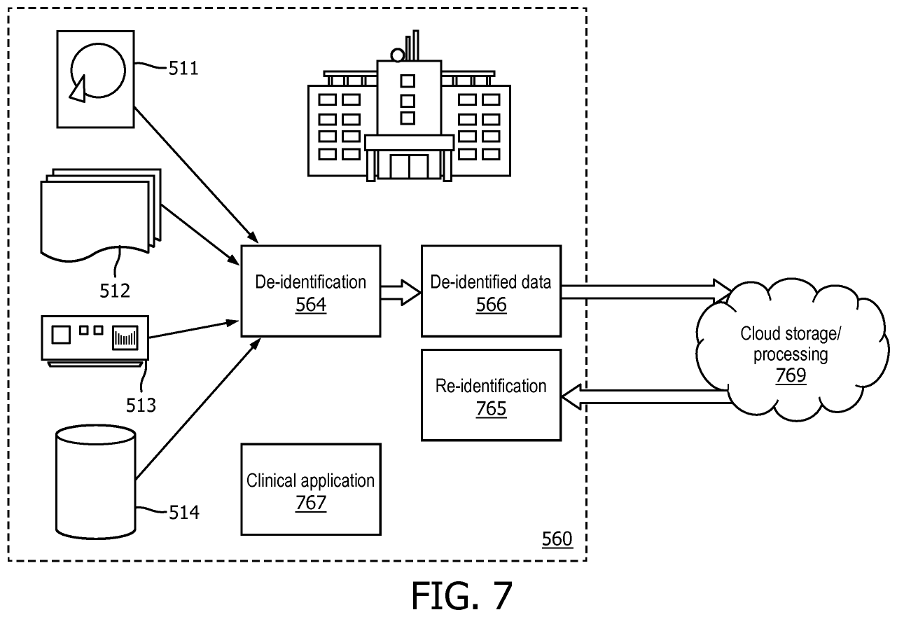

As noted previously, techniques described herein are not limited to the healthcare context, nor are they limited to providing de-identified data to outside entities for research or data analytics purposes. For example, techniques described herein may enable protected data (e.g., PHI) to be stored more safely outside of a secure environment, e.g., on a cloud infrastructure. Put another way, techniques described herein facilitate round-trip re-identification for cloud-based production applications. FIG. 7 depicts one example of how this may be implemented, and is similar to FIG. 5 in many respects.

In FIG. 7, de-identification module 564 once again may be configured to process raw data received from one or more data sources 511-514 to generate de-identified data 566. This de-identified data 566 may then be transferred, e.g., across one or more computing networks, to a cloud storage and/or processing infrastructure 769, which may include one or more server computers, such as one or more “blade” servers, that act upon the de-identified data in various ways. In some embodiments, a re-identification module 765 may be configured to retrieve de-identified data from the cloud storage/processing infrastructure, e.g., for use by one or more clinical applications 767 operating at hospital 560, and re-identify the data to its original form (e.g., using a persisted PHI lookup table). For example, clinical application 767 may be a CDS application that helps medical personnel make decisions based on re-identified data. One benefit of the process shown in FIG. 7 is to reduce perceived risk of cloud hosting or processing, while still benefiting from the scalable and virtually limitless resources of the cloud. This also allows for centralized management of primary application code, with the on-premises part of the application (e.g., 767) only consisting of the display logic and user interaction.

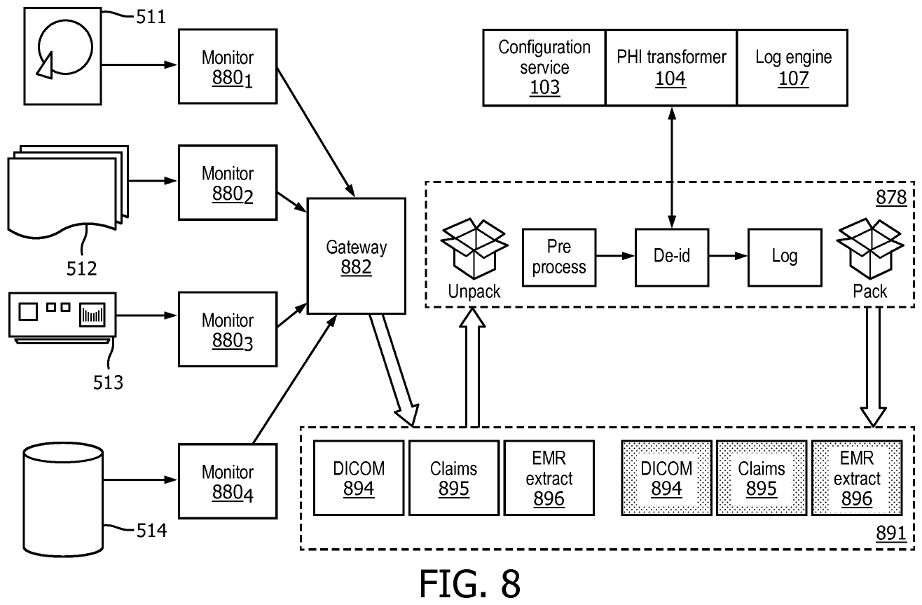

FIG. 8 depicts another example of an environment in which selected aspects of the present disclosure may be implemented, in accordance with various embodiments, in order to implement a data processing pipeline 878 configured with selected aspects of the present disclosure. FIG. 8 is similar to FIGS. 1, 5, and 7 in many respects, except that it includes more detail that may be implemented in selected embodiments. For example, the same data sources 511-514 present in FIGS. 5 and 7 are also present in FIG. 8.

In FIG. 8, a series of data monitors 880₁₋₄ may be employed to monitor or “listen” for data from the various data sources 511-514. Each data monitor 880 may listen for a particular type or types of data from a particular data source. In some implementations, data monitors 880₁₋₄ may be implemented as plug-ins for the overall structure, such that individual data monitors may be added, replaced, or removed as needed. While four data monitors 880₁₋₄ are depicted in FIG. 8, this is not meant to be limiting. There may be as many or as few data monitors 880 as there are different types of data and/or different data sources.

Data monitors 880 may take various forms, depending on the data type/source they “listen” to. For example, in some embodiments, one or more data monitors 880 may be configured as a PACs listener that receives, for instance, DICOM images and associated metadata. Additionally or alternatively, in some embodiments, one or more data monitors 880 may be configured as a time-based structure query language (“SQL”) query for data such as EMR extracts, claims, etc. Such a SQL query could be run periodically (e.g., every hour, day, week, five minutes, etc.) or on demand. Additionally or alternatively, in some embodiments, one or more data monitors 880 may be configured to listen to one or more filesystems for new files or objects. Additionally or alternatively, in some embodiments, one or more data monitors 880 may be configured to act as a REST, RMQ, or other interface for active transmissions.

As with FIG. 1, in FIG. 8, PHI transformer 104, configuration service module 103, and logging engine 107 are present and serve similar roles. For example, PHI transformer 104 may provide a centralized implementation of PHI policies. For example, it may provide the handlers described previously that are configured to process the individual data points, e.g., for de-identification, deduplication, redaction, substitution, ID hashing, time shifting (e.g., global time shifts, per-subject time shifts, per-encounter time shifts such as contextual time shifts, etc.), and so forth. In some embodiments, PHI transformer 104 may facilitate configurable noise addition to data, such as SALT.

Configuration service module 103 may once again be configured to facilitate versioned configuration, which in some embodiments may be recorded for provenance tracking purposes. Configuration service module 103 may in some cases provide a dynamically-generated central administration user interface. For example, a graphical user interface that is operable by one or more users to adjust configuration parameters may be customized to the particular installation/configuration. Additionally, in some embodiments, configuration service module 103 may facilitate schema-enforced configuration for a variety of services and/or policy handlers.

Logging engine 107 may be configured to store version data for provenance tracking, as well as to generate reports required, for instance, to audit the system. In some embodiments, logging engine 107 enables rapid inspection and review of data transmitted between various components (or to outside components such as research entities 670 or cloud infrastructure 769. Logging engine 107 also may be operable to uncover code and/or configuration parameters that are responsible for detected PHI leakage (described previously), and in some cases may be operable, e.g., alone or in conjunction with other components, to rescind output of code or a configuration that lead to detected leakage.

In various embodiments, protected data such as PHI may be processed by the components depicted in FIG. 8 as follows. First, a data monitor 880 discovers new data at its respective data source (e.g., 511-514). As an example, a data monitor 880 configured as a PACS listener may receive a set of new DICOM images, or a directory with new database extracts may be created. The data monitor 880 may send a message to a gateway service 882 (which may perform a similar role as message broker 101 in FIG. 1). This message may, in some embodiments, be a REST message, though this is not required. The REST message may include metadata (e.g. source hospital, other provenance info) and/or a payload, as was described previously with respect to FIG. 1. Gateway service 882 creates a new UUID for the data, creates a location in a storage area (“staging”) for the data, and responds to the requesting data monitor 880 with the staging location. The data monitor 880 may then copy the new data into the staging area provided by gateway service 882, and may notify gateway service 882 that the transfer is complete.

In some embodiments, gateway service 882 may verify the payload in the staging area. Assuming the data is verified (e.g., as non-corrupt, complete, etc.), the data may then be moved into a content repository 891 of the data pipeline 878, e.g., in a new subdirectory named with the assigned UUID. In some embodiments, gateway service 882 may send a REST call to the data pipeline 878, e.g., using technologies such as NiFi, to begin a job. In some embodiments, gateway service 882 may also provide provenance info and the location in the content repository 891 of the new data.

In various embodiments, data pipeline 878 may initiate an appropriate sub-pipeline based on the type of data. For example, DICOM data may trigger a DICOM processing flow, FHIR data may trigger a structured data flow, and so forth. In some embodiments, gateway service 882 (e.g., using NiFi) may create another UUID for output data, and may specify a new location (e.g., directory) in content repository 891 named with that new UUID. Gateway service 882 may then make a REST call to data pipeline 878, passing source UUID and new UUID as input and output locations. Gateway service 882 may then wait for completion.

Data pipeline 878 may unpack the data in the specified input location, de-identify it using one or more handlers (e.g., provided by PHI transformer 104), and may log various aspects of the processing, e.g., for downstream re-identification, auditing, and/or provenance tracking. While not specifically depicted in FIG. 8, in various embodiments, data pipeline 878 may include one or more de-identification modules similar to those (102) depicted in FIG. 1. One or more de-identification modules may load (or “unpack”) the data from the input location specified in the REST call, and then may use one or more handlers provided by PHI Transformer 104 to process dates, IDs, and other PHIs. In various embodiments, the one or more de-identification modules may then “pack” and/or save the de-identified output (e.g., 566) to the output location specified in the REST call. For example, DICOM, dates and IDs contained in metadata may be transformed using one or more handlers provided by PHI transformer 104. In some implementations, image data may be processed for text removal. Transformed metadata and image data may be recombined (e.g., “packed”) into a valid DICOM. Thus, as shown in FIG. 8, raw data such as DICOM data 894, claims data 895, and EMR extractions 896 (among others), which are represented by the “unfilled boxes,” may be processed and then stored the output location of content repository 891 as de-identified data, as represented by the corresponding shaded boxes.

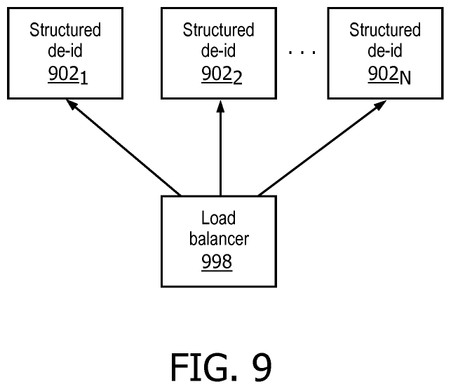

As noted elsewhere herein, the ability to handle large volumes of incoming data and to make optimal use of available resources by horizontal scaling is important. Accordingly, in some implementations, one or more de-identification modules (e.g., 102 in FIG. 1) may be controlled by a load balancing module to divide and conquer large data sets. Referring now to FIG. 9, a plurality of de-identification modules 902_{1-N}, which may be similar to modules 102_{1-N} in FIG. 1, are depicted under the control of a load balancer 998 (which may be implemented using any combination of hardware and software). In FIG. 9, the components may be stateless REST services, allowing for load balancing, rolling upgrades, etc., although this is not required. In some implementations, each de-identification module (e.g., 102, 902) may be implemented as a virtual machine that executes one or more handlers to completion, and then closes or executes another handler as needed.

In some implementations, a DICOM de-identification module (102/902, micro-service, remote service, etc.) may be implemented as a synchronous REST service in which the call returns when the study or batch of studies have completed. This can be scaled up to handle more data by way of load balancer 998, which may be configured to alternately call de-identification modules 902, e.g. in a round robin or other distribution. One advantage of this approach is that it is simple for the caller to consume, and no polling or other process checking is required. In some embodiments, load balancer 998 may assign different priorities to different de-identification modules 902, e.g., based on factors such as computation resources required, time running, time left to complete, amount of data to process, etc. For example, if a particular de-identification module 902 is assigned a computationally expensive task, that de-identification module 902 may be assigned greater priority than others, and therefore may be afforded more computing cycles, cloud-based resources, GPU cycles, etc.

An alternate approach may be used in various embodiments in which the REST service is separated from the de-identification modules 102/902. For example, the message broker 101 of FIG. 1 and the gateway service 882 only serve to add a message to a worker queue (which may be handled, for instance, by RabbitMQ) or to retrieve job status. In some embodiments, a REST call made to message broker 101 or gateway service 882 may be asynchronous and return immediately with the job id, which may be used by the caller to poll for status. A set of de-identification modules 102/902 consume the queue, each taking the next available job, processing the input data associated with the job, and publishing the job's status to the message broker 101/gateway service 882.

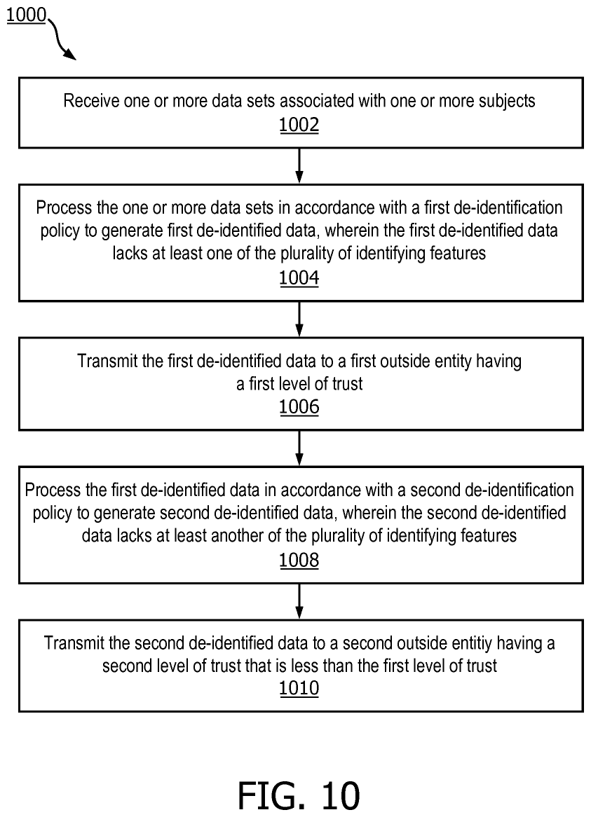

FIG. 10 depicts an example method 1000 for practicing selected aspects of the present disclosure, particularly progressive de-identification, in accordance with various embodiments. For convenience, the operations of the flow chart are described with reference to a system that performs the operations. This system may include various components of various computer systems, including components depicted in FIGS. 1 and 5-8. Moreover, while operations of method 1000 are shown in a particular order, this is not meant to be limiting. One or more operations may be reordered, omitted or added.

At block 1002, the system may receive one or more data sets associated with one or more subjects, e.g., in varying forms such as JSON, CSV, database extracts, DICOM (e.g., metadata or image data/image data extracts), etc. In various embodiments, each of the one or more data sets may contain a plurality of data points associated with a respective subject of the one or more subjects. The plurality of data points may include a plurality of identifying (or at least potentially identifying) features that are usable to identify the one or more subjects. For example, each data set may include, for a respective subject, one or more identifiers (e.g., social security number, driver's license number, medical record number, etc.), one or more location data types (e.g., ZIP code, city, state, etc.), one or more dates/times (e.g., birthday, hospital admission, hospital encounter, etc.), and so forth.

At block 1004, the system may process the one or more data sets in accordance with a first de-identification policy to generate first de-identified data (e.g., 672₁). The resulting first de-identified data may lack at least one of the plurality of identifying features. The first de-identification policy may be a government-imposed law regulation (e.g., HIPAA), a master research agreement, a business-to-business agreement, a university-to-business agreement (or vice versa), and so forth. In various embodiments, processing the one or more data sets in accordance with the first de-identification policy may ensure that a first outside entity (e.g., 670₁) that desires/requested the data only has the data that it “needs to know,” and is not provided potentially identifying data (e.g., PHI) that is unnecessary for its purposes. The first outside entity may be, for instance, a researcher, a university, another hospital, a private enterprise, a laboratory, a government agency, etc.

At block 1006, the first de-identified data may be transmitted, e.g., over one or more computing networks, to a computing system operated by the first outside entity having a first level of trust. For example, the first outside entity may be a research entity that is deemed relatively trustworthy in that they can be relied upon to ensure that the de-identified data is secured. That way, any remaining identifying or potentially identifying features in the one or more data sets may still be protected from unauthorized parties, at least to an acceptable degree.

At block 1008, the system may process the first de-identified data in accordance with a second de-identification policy to generate second de-identified data (e.g., 672₂). The resulting second de-identified data may lack at least another of the plurality of identifying features mentioned previously, in addition to those identifying feature(s) that were already addressed at block 1004. Notably, processing the first de-identified data in accordance with the second de-identification policy, rather than the original raw data, may save considerable time and/or computing resources because at least some data points may already be de-identified. This is particularly true if removal of the applicable identifying features during the operations of block 1004 required considerable resources. Such might be the case where, for instance, the identifying features had to first be extracted/detected in DICOM images, and/or free text had to be analyzed, e.g., using natural language processing, to flag potentially identifying data point(s) for obfuscation/removal.

At block 1010, the system may transmit, e.g., over one or more computing networks, the second de-identified data to a computing system operated by a second outside entity (e.g., 670₂) having a second level of trust that is less than the first level of trust. The second outside entity may be, for instance, a private business or commercial enterprise for which internal security measures may not necessarily be known. In such case, it may be safer to ensure that data sent to the second outside entity is thoroughly scrubbed (at least more than it was for the first outside entity), reducing the need to rely on the second outside entity's own security measures.

While data is processed and distributed to two outside entities in FIG. 10, this is not meant to be limiting. Progressive de-identification techniques described herein may be used to distribute data that is de-identified at any number of levels to any number of outside entities. And it is not required that each outside entity receive its de-identified data at or near the same time. For example, the first outside entity may request/receive the first de-identified data weeks, months, or even years before the second outside entity receives the second de-identified data. Additionally, in some embodiments it is possible to generate and distribute more heavily de-identified data first (e.g., to the second outside entity), and then later re-identify at least part of the second de-identified data (if necessary) to generate the first de-identified data for the first outside entity.

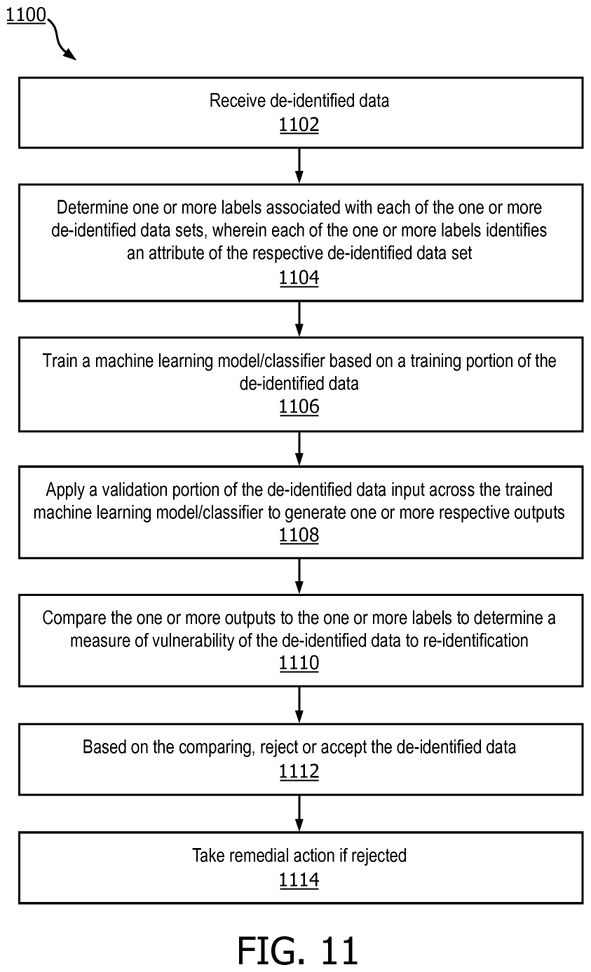

FIG. 11 depicts an example method 1100 for practicing selected aspects of the present disclosure, in accordance with various embodiments. For convenience, the operations of the flow chart are described with reference to a system that performs the operations. This system may include various components of various computer systems, including components depicted in FIGS. 1 and 5-8. Moreover, while operations of method 1100 are shown in a particular order, this is not meant to be limiting. One or more operations may be reordered, omitted or added.

At block 1102, the system, e.g., by way of data security officer 568, may receive de-identified data (e.g., 566). The de-identified data may include one or more de-identified data sets associated with one or more subjects that are generated from one or more raw data sets associated with the one or more subjects. The raw data sets may come from, for instance, one or more data sources (e.g., 111-112, 511-514). Each of the one or more raw data sets may contain one or more data points associated with a respective subject of the one or more subjects, such as an identify, location, age, weight, and one or more physiological measurements or data points. In some embodiments, the one or more data points may include one or more identifying features (e.g., identifier such as social security number, date/time such as birthdate, hospital admission, etc.) that are usable to identify the respective subject. At least some of the one or more identifying features are absent from or obfuscated in the de-identified data, e.g., by virtue of having been processed using techniques described herein.

At block 1104, the system may determine one or more labels associated with each of the one or more de-identified data sets. Each of the one or more labels may identify an attribute of the respective de-identified data set, such as which version of a handler was used to process one or more data points, which configuration was used (e.g., what type of hashing function), an indication of whether a date or time (e.g., birthdate, hospital encounter date, admission date, etc.) occurred before or after some date or time threshold (which may be arbitrarily selected, e.g., based on a temporal midpoint of a subject's data timeline), a date or time shift, whether a date or time shift was applied, and so forth.

At block 1106, the system may train a machine learning model/classifier. The classifier can learn weights in a training stage utilizing one or more machine learning algorithms as appropriate to the classification task in accordance with many embodiments including linear regression, logistic regression, linear discriminant analysis, principal component analysis, classification trees, regression trees, naïve Bayes, k-nearest neighbors, learning vector quantization, support vector machines, bagging forests, random forests, boosting, AdaBoost, neural network(s), etc. As noted previously, in some embodiments, a first “training” portion of the de-identified data may be used for training (e.g., 70% or some other fraction).

Training at block 1106 may include, for instance, applying the training portion of the de-identified data (e.g., 70% of the data) as input across the model to generate output, and comparing the output to labels associated with the training portion. Based on the comparison, various training techniques (e.g., gradient descent, back propagation, etc.) may be employed to alter one or more parameters of the model/classifier. Once trained, the machine learning model/classifier may be able to predict the labeled attribute in unlabeled data to a certain degree of accuracy. If the degree of accuracy is too high, the de-identified data (and the techniques/handlers/configuration) used to process it may be tainted or vulnerable to leakage. The degree of accuracy will be determined at block 1110, which is described below.

At block 1108, the system may apply a validation portion of the de-identified data (e.g., the remaining 30%) as input across the trained machine learning model to generate one or more respective outputs. Each of the one or more respective outputs may be indicative of whether the respective de-identified data set has the attribute. As noted above, at block 1110, the system may compare the one or more outputs to the one or more labels (associated with the validation portion) to determine a measure of vulnerability of the de-identified data to re-identification. At block 1112, the system may, based on the comparing, reject or accept the de-identified data. For example, in some embodiments, if the accuracy of the trained machine learning model/classifier exceeds some threshold, such as AUC of 0.8, then the de-identified data may be rejected.

At block 1114, various remedial actions may be taken, such as notifying a human version of data security office 568 via output such as an audible or visible alert, a text message, an email, etc. In some embodiments, the individual data points found most important by the trained machine learning model/classifier may be output to data security officer 568 for inspection. These problematic and/or highly influential data points may be removed, or additional processing may be applied as needed. For example, a stronger hash algorithm may be used, or a context date/time shift may be applied instead of a general date/time shift.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

1. A method implemented using one or more processors, comprising: receiving de-identified data, wherein the de-identified data includes one or more de-identified data sets associated with one or more subjects that is generated from one or more raw data sets associated with the one or more subjects, each of the one or more raw data sets containing one or more data points associated with a respective subject of the one or more subjects, wherein the one or more data points include one or more identifying features that are usable to identify the respective subject, and wherein at least some of the one or more identifying features are absent from or obfuscated in the de-identified data;
determining one or more labels associated with each of the one or more de-identified data sets, wherein each of the one or more labels identifies an attribute of the respective de-identified data set;
applying at least some of the one or more de-identified data sets as input across a trained machine learning model to generate one or more respective outputs, wherein each of the one or more respective outputs is indicative of whether the respective de-identified data set has the attribute;
comparing the one or more outputs to the one or more labels to determine a measure of vulnerability of the de-identified data to re-identification; and
based on the comparing, rejecting or accepting the de-identified data.

2. The method of claim 1, wherein the attribute comprises a version of one or more handlers used to process the one or more raw data sets.

3. The method of claim 1, wherein each of the one or more labels indicates whether a date or time data point in the respective de-identified data set occurs before or after a threshold date or time.

4. The method of claim 1, wherein the one or more de-identified data sets comprise a plurality of de-identified data sets.

5. The method of claim 4, wherein the at least some of the plurality of de-identified data sets comprise a training portion of the plurality of de-identified data sets, and the method further comprises training the machine learning model using the training portion of the plurality of de-identified data sets, wherein the applying comprises applying a remaining validation portion of the plurality of de-identified data sets as input across the trained machine learning model as validation of the training.

6. The method of claim 1, wherein the one or more subjects comprise one or more patients, and the one or more raw data sets associated with the one or more subjects include medical records associated with the one or more patients.

7. The method of claim 1, wherein the trained machine learning model includes a random forest or Ada Boost component.

8. At least one non-transitory computer-readable medium comprising instructions that, in response to execution of the instructions by one or more processors, cause the one or more processors to perform the following operations:
receiving de-identified data, wherein the de-identified data includes one or more de-identified data sets associated with one or more subjects that is generated from one or more raw data sets associated with the one or more subjects, each of the one or more raw data sets containing one or more data points associated with a respective subject of the one or more subjects, wherein the one or more data points include one or more identifying features that are usable to identify the respective subject, and wherein at least some of the one or more identifying features are absent from or obfuscated in the de-identified data;
determining one or more labels associated with each of the one or more de-identified data sets, wherein each of the one or more labels identifies an attribute of the respective de-identified data set;
applying at least some of the one or more de-identified data sets as input across a trained machine learning model to generate one or more respective outputs, wherein each of the one or more respective outputs is indicative of whether the respective de-identified data set has the attribute;
comparing the one or more outputs to the one or more labels to determine a measure of vulnerability of the de-identified data to re-identification; and
based on the comparing, rejecting or accepting the de-identified data.

9. The at least one non-transitory computer-readable medium of claim 8, wherein the attribute comprises a version of one or more handlers used to process the one or more raw data sets.

10. The at least one non-transitory computer-readable medium of claim 8, wherein each of the one or more labels indicates whether a date or time data point in the respective de-identified data set occurs

11. The at least one non-transitory computer-readable medium of claim 8, wherein the one or more de-identified data sets comprise a plurality of de-identified data sets.

12. The at least one non-transitory computer-readable medium of claim 11, wherein the at least some of the plurality of de-identified data sets comprise a training portion of the plurality of de-identified data sets, and the computer-readable medium further comprises instructions for training the machine learning model using the training portion of the plurality of de-identified data sets, wherein the applying comprises applying a remaining validation portion of the plurality of de-identified data sets as input across the trained machine learning model as validation of the training.

13. The at least one non-transitory computer-readable medium of claim 8, wherein the one or more subjects comprise one or more patients, and the one or more raw data sets associated with the one or more subjects include medical records associated with the one or more patients.

14. The at least one non-transitory computer-readable medium of claim 8, wherein the trained machine learning model includes a random forest or AdaBoost component.

15. A system comprising one or more processors and memory operably coupled with the one or more processors, wherein the memory stores instructions that, in response to execution of the instructions by one or more processors, cause the one or more processors to perform the following operations:
receiving de-identified data, wherein the de-identified data includes one or more de-identified data sets associated with one or more subjects that is generated from one or more raw data sets associated with the one or more subjects, each of the one or more raw data sets containing one or more data points associated with a respective subject of the one or more subjects, wherein the one or more data points include one or more identifying features that are usable to identify the respective subject, and wherein at least some of the one or more identifying features are absent from or obfuscated in the de-identified data;
determining one or more labels associated with each of the one or more de-identified data sets, wherein each of the one or more labels identifies an attribute of the respective de-identified data set;
applying at least some of the one or more de-identified data sets as input across a trained machine learning model to generate one or more respective outputs, wherein each of the one or more respective outputs is indicative of whether the respective de-identified data set has the attribute;
comparing the one or more outputs to the one or more labels to determine a measure of vulnerability of the de-identified data to re-identification; and
based on the comparing, rejecting or accepting the de-identified data.

16. The system of claim 15, wherein the attribute comprises a version of one or more handlers used to process the one or more raw data sets.

17. The system of claim 15, wherein each of the one or more labels indicates whether a date or time data point in the respective de-identified data set occurs before or after a threshold date or time.

18. The system of claim 15, wherein the one or more de-identified data sets comprise a plurality of de-identified data sets.

19. The system of claim 18, wherein the at least some of the plurality of de-identified data sets comprise a training portion of the plurality of de-identified data sets, and the system further comprises instructions for training the machine learning model using the training portion of the plurality of de-identified data sets, wherein the applying comprises applying a remaining validation portion of the plurality of de-identified data sets as input across the trained machine learning model as validation of the training.

20. The system of claim 15, wherein the one or more subjects comprise one or more patients, and the one or more raw data sets associated with the one or more subjects include medical records associated with the one or more patients.
